(19) United States
(12) Patent Application Publication
(10) Pub. No.: US 2026/0315509 A1
(43) Pub. Date: Oct. 15, 2026

(54) 3-Fucosyllactose and butyrate for food allergy

(71) Applicant: N.V. Nutricia, Zoetermeer (NL)

(72) Inventors: Belinda Van 't Land - Potappel, Utrecht (NL); Atanaska Ivanova Kostadinova, Utrecht (NL); Linette Eustachia Maria Willemsen, Utrecht (NL); Marit Zuurveld, Utrecht (NL)

(73) Assignee: N.V. Nutricia, Zoetermeer (NL)

(21) Appl. No.: 19/568,884

(22) Filed: Mar. 17, 2026

Related U.S. Application Data
(63) Continuation of application No. PCT/EP2024/077139, filed on Sep. 26, 2024.

(30) Foreign Application Priority Data
Sep. 26, 2023 (EP) 23199803.0

(51) Int. Cl. A61K 31/702; A23L 33/00; A61K 9/00; A61K 31/19; A61K 35/00; A61K 35/745

(52) U.S. Cl. CPC A61K 31/702; A23L 33/40; A61K 9/0095; A61K 31/19; A61K 35/745; A61K 2035/115

(57) ABSTRACT

The invention pertains to nutritional compositions and uses thereof comprising digestible carbohydrates, lipids, protein, 3-fucosyllactose (3-FL) and a source of butyrate suitable for providing nutrition to an infant or young child suffering from or at risk of developing a food allergy, preferably cow's milk protein or hen's egg allergy, wherein the protein is or essentially consists of hydrolysed protein and/or free amino acids and wherein the butyrate is provided in the form of dietary butyrate and/or by non-digestible saccharides selected non-digestible polysaccharides, non-digestible oligosaccharides or mixtures thereof capable of undergoing microbial fermentation in the gastrointestinal tract to produce butyrate.

### FIELD OF THE INVENTION

The present invention relates to composition comprising at least 3-fucosyllactose (3-FL) and a source of butyrate and applications of the compositions in food allergy prevention and immune tolerance induction in infants.

### BACKGROUND OF THE INVENTION

Human milk is the preferred food for infants. Human milk provides several bioactive factors that benefit the relatively immature immune system and the intestinal health of neonates early in life. Human milk fed infants have a lower incidence of infections and allergies than formula fed infants. Many components in human milk, including immunoglobulins (such as sIgA), interleukin (IL)-1, IL-6, IL-8, IL-10, interferon-y (IFN-y), immunocompetent cells, transforming growth factor-β (TGF-β), lactoferrin, nucleotides and human milk oligosaccharides (HMOs) are thought to play a role in protection against infection with pathogens. Additionally intestinal maturation and development of the microbiota in human milk fed infants is considered optimal.

When breastfeeding is not possible or the mother chooses not to breastfeed, infant formulas are commercially available that are suitable as a complete nutrition. Usually, these formulas are based on intact cow's milk protein, in particular whey protein and casein.

Food allergens are among the first allergens that infants encounter in their early life: typically, cow's milk proteins may be encountered by infants not receiving exclusive breast feeding. In early life, cow's milk allergy (CMA) is the most dominant food allergy; About 2 to 3% of infants are allergic to cow's milk protein and the prevalence increased over the last decades [Tsuge et al, “Current insights into atopic march,” Children, 2021]. Besides acute clinical manifestations, which can be severe, CMA in early life can also have long-lasting effects, including delays in growth and development, as well as increased risk for the development of atopic diseases later in life. Hence, strategies to treat or prevent CMA are of major importance.

Dietary proteins such as cow's milk proteins and hens egg proteins are presented to the immune system via the gastrointestinal tract and the normal response during immune system maturation would be to elicit a tolerogenic immune response to the ingested nutrients. This response is called oral immune tolerance or oral tolerance. The induction of oral immune tolerance is especially relevant for infants, who after birth are exposed for the first time to dietary proteins and have to adapt to this. If oral immune tolerance in infants is not properly established, food allergic sensitization may occur.

The standard dietary management of for instance CMA in infants and children is allergen avoidance through elimination of the allergen from the diet. A variety of formulas have been developed for the elimination of cow's milk protein. Extensively hydrolysed formulas (eHFs) are recommended for infants with mild to moderate CMA, whereas for infants with severe CMA and for infants that either do not tolerate eHFs or in which eHFs fail to resolve CMA symptoms, amino acid-based formulas (AAFs) are recommended.

Current symptom management in food allergic patients consists of active avoidance of the allergens, however there is an increasing interest in limiting the development of a food allergy by preventing allergic sensitization. Allergic sensitization in childhood, especially in early childhood and especially to food allergens, is critical and of the highest interest as the development of an “allergic phenotype” or “atopy” has been shown to facilitate subsequent sensitization to other allergens. Hence allergies in childhood can be the first step leading to multiple allergies later in life, a process commonly referred to as the “Atopic March”.

The immune system of infants is actively developing all along the first few years of life and this immune maturation involves the development of type 1 immunity at the cost of type 2 immunity. Acting on, preventing, avoiding, managing, reducing, or modulating the allergic reactions in such young patients can influence their allergic profile, not only in the short term but also longer term for later in life. Prevention of the onset of allergy is therefore an important aspect when considering development of formula milk concepts for early life.

HMOs (human milk oligosaccharides) have become the subject of much interest in recent years due to their roles in numerous biological processes including immune related processes occurring in humans. Mammalian milk contains at least 200 of these complex oligosaccharides (Kunz C et al. Annu Rev Nutr 20: 699-722, 2000).

Exposure to human milk oligosaccharides (HMOs) via breastmilk contributes to maturation of the immune system, either directly or e.g. via interactions with intestinal epithelial cells (IEC), and/or by other bioactive compounds which arise after fermentation of HMOs by intestinal microbial species into other bioactive compounds such as short chain fatty acids (SCFA) including butyric acid [Singh, et al “Recent understanding of human milk oligosaccharides in establishing infant gut microbiome and roles in immune system,” Food Res. Int., 2021]. Paparo et al Allergy. 2021; 76:1398-1415 describes a beneficial role of gut microbiota-derived butyrate on allergic responses. Corona et al Children 2021, 8; 804 provides an overview of structure-function relation of various HMOs.

In early life, HMOs present in breast milk, form the major source of non-digestible oligosaccharides (NDOs). Various immunomodulatory properties have been attributed to HMOs and SCFA, including both direct effects on epithelial cells and immune cells and indirect effects via the microbiome [Zuurveld, et al., “Immunomodulation by Human Milk Oligosaccharides: The Potential Role in Prevention of Allergic Diseases,” Front. Immunol., 2020], such as induction of mucus production, decreased epithelial barrier permeability, interaction with G protein-coupled receptors (GPRs), inhibition of histone deacetylases (HDACs), and the NF-κB pathway in IEC, as well as improved regulatory T cell (Treg) formation. Many of these pathways are also actively involved in allergic diseases.

Functionally, allergy results from a lack of tolerance induction towards allergens and an inappropriate T-helper type 2 (Th2) immune response against otherwise generally innocuous proteins. Evidence points to the link between the intestinal microbiota and Th2 responses suggesting that the intestinal microbiota is involved in the development of systemic Th2 responses by supporting the induction of immune homeostasis. Hence, an imbalance (dysbiosis) in the intestinal microbiota (and lower levels of bacterial fermentation products such as SCFA amongst which butyrate), may cause impaired capacity for proper development of immune homeostasis and tolerance; increasing the risk for the development of allergies.

WO 2006/115412 A1 relates to a liquid nutritional composition for improving intestinal barrier function and for preventing allergy. The composition comprises lipids with short chain fatty acyl chains and non-digestible, fermentable saccharides.

Zuurveld et al Biomolecules 2023, 13(2), 263 describes 2′-FL and 3-FL may have immunomodulatory properties in neonatal immune maturation by promoting enhanced type 1 and regulatory mediator secretion in an allergy model.

WO2011/008086 describes nutritional compositions comprising 2′-FL and beta-galacto-oligosaccharides amongst others for use in the treatment of immune disorders. WO2018/210807 A1 describes nutritional compositions with galacto-oligosaccharides and HMOs and the use in modifying the gut microbiome. WO2012/158517 A1 in turn describes 2′-FL, 3-FL and LDFT for the stimulation of the growth of bacteria in the human gastrointestinal tract.

WO2022/253980 A1 describes age-specific infant formulae comprising 3-FL, 3′-SL, DFL and 2′-FL, while WO2021116236 A1 describes an age-specific nutritional composition system comprising for different infant age-groups a different amount of 2′-FL, 3-FL, LNT, 6′-SL and 3′-SL.

WO 2020/245311 A1 relates to nutritional composition comprising 2′-fucosyllactose and dietary butyrate for the improvement of intestinal health, in particular for treatment or preventing allergy and inducing oral tolerance to allergens.

WO2022/161865 A1 describes compositions comprising bifidobacteria and an HMO mixture consisting of 2′-FL, DFL, LNT, 6′-SL and 3′-SL for preventing and/or treating allergy by increasing intestinal short chain fatty acid production.

WO20201207641 A1 describes sources of butyrate containing a glycerol backbone with one or more butyrate moieties for use in allergic disease. WO2018/210805 A1 describes preterm infant formulae containing butyrate.

US2014248415 A1 describes several examples of HMOs mixtures, including both 2′-FL and LNnT in various ratios for various health benefits including immune system maturation and allergy prevention.

Various approaches have been proposed to lower the risk of allergy development in early life and/or improve induction of oral tolerance to food allergens in infants and young children, yet there is an ongoing need to improve oral tolerance development and prevent and/or treat food allergies.

### SUMMARY OF THE INVENTION

The inventors set out to assess the interaction between butyrate and the fucosylated human milk oligosaccharides, 2′-fucosyllactose (2′-FL) and 3-fucosyllactose (3-FL), on intestinal epithelial function upon ovalbumin (OVA) induced type 2 activation of human intestinal epithelial cells (IEC) and subsequent dendritic cell (DC) maturation and T-helper cell functioning in a sequential mucosal co-culture model. The inventors found that a combination of the nutritional ingredients 3-FL and butyrate have a beneficial effect on immune maturation and prevention of allergy prone type 2 response, in particular in a food allergen exposed condition which may lead to the prevention of food allergy. It was found that the response of the immune cells is different when both 3-FL and butyrate are provided compared to when only one of these ingredients is present. The mixture of 3-FL and butyrate has been shown to have a beneficial and synergistic effect on modulation of type 2 immunity, by promoting the Th1 response while suppressing the Th2 response as shown by the increased ratio of IFNγ/IL13 as secreted by T helper cells. 3-FL and butyrate together beneficially promote the immune balance in favour of a type 1 over a type 2 response skewing away from the allergic phenotype. This is indicative of beneficial uses of 3-fucosyllactose (3-FL) and a source of butyrate suitable for providing nutrition to an infant or young child suffering from or at risk of developing or suffering from a food allergy, preferably cow's milk protein or hen's egg allergy.

### LIST OF FIGURES

The present invention will be discussed in more detail below, with reference to the attached figures.

FIG. 1 A) shows the effect of 2′-FL, 3-FL, butyrate, and mixtures thereof on IFN-γ production by naïve T-helper cells cocultured with dendritic cells [DCs] primed by OVA exposed IEC. FIG. 1 B) shows the effect of 2′-FL, 3-FL, butyrate, or mixtures thereof on IL-13 production of naïve T-helper cells cocultured with DCs primed by OVA exposed IEC. In these experiments IEC were pre-exposed to 2′-FL, 3-FL, butyrate, or mixtures prior to OVA exposure.

FIG. 2 shows the Th1 to Th2 ratio as illustrated by the IFN-γ to IL-13 ratio.

### LIST OF PREFERRED EMBODIMENTS

1. A nutritional composition comprising digestible carbohydrates, lipids, protein, 3-fucosyllactose (3-FL) and a source of butyrate suitable for providing nutrition to a human subject, preferably an infant or young child suffering from or at risk of developing a food allergy, preferably cow's milk protein or hen's egg allergy, wherein the protein is or essentially consists of hydrolysed protein and/or free amino acids and wherein the source of butyrate is provided in the form of dietary butyrate and/or by non-digestible saccharides capable of undergoing microbial fermentation in the gastrointestinal tract to produce butyrate,
wherein non-digestible saccharides are selected from non-digestible polysaccharides [NDP], non-digestible oligosaccharides [NDO] or mixtures thereof, and
wherein the non-digestible saccharides are plant-derived, crustacean-derived and/or microorganism derived.
2. The nutritional composition according to embodiment 1, in the form of a ready to drink liquid or in the form of a powder that upon reconstitution with water is a ready to drink liquid nutritional composition, wherein the 3-FL is present in an amount of 10 mg to 1 g 3-FL per 100 ml, more preferably 15 mg to 0.5 g 3-FL, even more preferably 20 mg to 0.2 g 3-FL per 100 ml nutritional composition.
3. The nutritional composition according to embodiment 1 or 2 wherein the butyrate is provided in the form of dietary butyrate and/or by non-digestible saccharides capable of undergoing microbial fermentation in the gastrointestinal tract to produce butyrate, wherein the nutritional composition comprises 10 mg to 175 mg dietary butyrate per 100 ml nutritional composition and/or 1 mg to 1.0 g non-digestible saccharides per 100 ml nutritional composition.
4. The nutritional composition according to any of the preceding embodiments, wherein the source of butyrate comprises dietary butyrate, wherein the sources of dietary butyrate are selected from the group consisting of food-grade tributyrin, anhydrous milk fat, butter oil, and microbial fermentation derived products.
5. The nutritional composition according to any of the preceding embodiments, wherein the source of butyrate comprises non-digestible oligosaccharides selected from the group consisting of fructo-oligosaccharides, xylooligosaccharides, arabino-oligosaccharides, arabinogalacto-oligosaccharides, gluco-oligosaccharides, glucomanno-oligosaccharides, galactomanno-oligosaccharides, mannan-oligosaccharides, chito-oligosaccharides, uronic acid oligosaccharides, and mixtures thereof.
6. The nutritional composition according to any of the preceding embodiments, wherein the source of butyrate comprises non-digestible polysaccharides selected from the group consisting of low viscosity pectin, fructo-polysaccharides, oat beta-glucan, soy fiber, resistant starch, acacia gum, cellulose, arabinoxylan, xanthan gum, locust bean gum and/or resistant starch.
7. The nutritional composition according to any of the preceding embodiments, wherein the source of butyrate comprises a mixture of non-digestible polysaccharides and non-digestible oligosaccharides, preferably a mixture of short-chain fructoologosaccharide (scFOS) and long-chain oligosaccharides (IcFOS), more preferably a mixture of scFOS and IcFOS in a weight ratio of 9:1.
8. The nutritional composition according to any of the preceding embodiments, wherein the source of butyrate comprises milk fat and/or fructo-oligosaccharides, preferably a mixture of short-chain fructo-oligosaccharides and long chain fructo-oligosaccharides.
9. The nutritional composition according to any one of the preceding embodiments, which is an infant formula, a follow-on formula or young child milk.
10. The nutritional composition according to any one of the preceding claims further comprising Bifidobacterium, preferably B. breve.
11. The nutritional composition according to any one of the preceding embodiments, wherein the protein is or essentially consists of hydrolysed whey protein or hydrolysed rice protein.
12. The nutritional composition according to any one of the preceding embodiments, for use in the dietary management of an infant or young child suffering from or at risk of food allergy, preferably hen's egg protein allergy or cow's milk protein allergy, more preferably cow's milk protein allergy.
13. The nutritional composition according to any one of embodiments 1-12, for use in the treatment or prevention of food allergy, preferably hen's egg protein allergy or cow's milk protein allergy, more preferably cow's milk protein allergy.
14. The nutritional composition according to any one of the preceding embodiments for use in the treatment or prevention of food allergy selected from stimulating the immune function, promoting immune maturation, reducing and/or depressing the Th2 response, improving and/or promoting the Th1 response, preferably for use in increasing the Th1:Th2 balance.
15. Use of lipids, digestible carbohydrates and protein in the manufacture of a nutritional composition for the dietary management of a human subject, preferably an infant or young child that suffers from or is at risk of allergy, preferably a human subject, preferably an infant or young child, that is at risk of or suffers from food allergy, preferably hen's egg or cow's milk protein allergy, wherein the protein is or essentially consists of hydrolysed protein and/or free amino acids and wherein the nutritional composition comprises a combination of 3-FL and a source of butyrate.
16. Method of treating or preventing food allergy in an infant or young child by modulating immune maturation, the method comprising administering to the infant or young child a nutritional composition as defined in any one of embodiments 1 to 11.
17. The nutritional composition according to any one of embodiments 1 to 11, wherein the source of butyrate is not 3-FL.
18. The nutritional composition according to any one of embodiments 1 to 11, wherein the nutritional composition is a hypoallergenic nutritional composition.

### DETAILED DESCRIPTION OF THE INVENTION

The present invention thus concerns a nutritional composition comprising digestible carbohydrates, lipids, protein, 3-fucosyllactose and a source of butyrate suitable for providing nutrition to a human subject, preferably an infant or young child at risk of or suffering from food allergy, in particular cow's milk protein allergy or hen's egg allergy, wherein the protein is or essentially consists of hydrolysed protein and/or free amino acids. The nutritional composition according to the invention is a hypoallergenic nutritional composition.

The present invention is in particular advantageous in so far as it provides an immune maturation promoting and a food allergy preventing effect, while the risk of provoking food allergic responses is low when using the protein component that is, or essentially consists of hydrolysed protein and/or free amino acids combined with an immunomodulatory component being 3-FL and a separate source of butyrate according to the present invention.

The present nutritional composition may be in the form of a ready to drink liquid or in the form of a powder that upon reconstitution with water is a ready to drink liquid. The nutritional composition is a synthetic nutritional composition and is not human breast milk.

The present invention essentially relates to a nutritional composition for human subjects, preferably infants or young children, comprising a combination of
a. 3-fucosyllactose (3-FL), and
b. butyrate, wherein butyrate is provided in the form of dietary butyrate and/or by non-digestible saccharides selected from non-digestible polysaccharides, non-digestible oligosaccharides, or mixtures thereof capable of undergoing microbial fermentation in the gastrointestinal tract to produce butyrate, said wherein the non-digestible saccharides are preferably plant-derived, crustacean-derived and/or microorganism derived.

The present invention further concerns a nutritional composition comprising digestible carbohydrates, lipids, and protein, wherein the protein is or essentially consists of hydrolysed protein and/or free amino acids and wherein the nutritional composition comprises a combination of 3-FL and a source of butyrate, for use in the dietary management of a human subject, preferably an infant or young child, that is at risk of or suffers from food allergy, preferably an infant or young child that suffers from food allergy, in particular cow's milk protein and/or hen's egg allergy. In the context of the invention 3-FL is an immunomodulatory component and not the separate source of butyrate. The source of butyrate is understood to be a separate source than 3-FL. The use in the dietary management comprises prevention of the development of food allergy. In some aspects, the use in the dietary management of a human subject, preferably an infant or young child that is at risk of or suffers from allergy comprises accelerated outgrowth of said food allergy.

This can also be worded as a method for the dietary management of a human subject, preferably an infant or young child, that is at risk of or suffers from food allergy, preferably an infant or young child that suffers from food allergy, in particular cow's milk protein and/or hen's egg allergy comprising administering to the human subject, preferably an infant or young child, a nutritional composition comprising digestible carbohydrates, lipids, and protein, wherein the protein is or essentially consists of hydrolysed protein and/or free amino acids and wherein the nutritional composition comprises a combination of 3-FL and a source of butyrate.

The invention can also be worded as the use of lipids, digestible carbohydrates and protein in the manufacture of a nutritional composition for the dietary management of a human subject, preferably an infant or young child, at risk of or that suffers from food allergy, preferably an infant or young child that is at risk of or suffers from food allergy, in particular cow's milk protein and/or hen's egg allergy, wherein the protein is or essentially consists of hydrolysed protein and/or free amino acids and wherein the nutritional composition comprises a combination of 3-FL and a source of butyrate.

The present invention further concerns a nutritional composition comprising digestible carbohydrates, lipids, and protein, wherein the protein is or essentially consists of hydrolysed protein and/or free amino acids and wherein the nutritional composition comprises a combination of 3-FL and a source of butyrate, for use in the promotion of immune maturation and/or prevention of the development of food allergy in a human subject, preferably an infant or young child, that is at risk of or suffers from food allergy, in particular cow's milk protein or hen's egg allergy.

Furthermore, the present invention concerns use of a combination of 3-FL and a source of butyrate in a hypoallergenic nutritional composition that is suitable for a human subject, preferably an infant or young child, that is at risk of or suffers from food allergy and more particularly cow's milk protein or hen's egg allergy, wherein the hypoallergenic nutritional composition comprises digestible carbohydrates, lipids and protein, wherein the protein is or essentially consists of hydrolysed protein and/or free amino acids.

In a preferred embodiment the protein source consists essentially of free amino acids. In other words, in some aspects, the nutritional composition is preferably an amino acid-based nutritional composition for use in immune system maturation and the treatment and prevention of food allergy.

### Definitions

In the context of the present invention the term “prevention” means “reducing the risk of (occurrence)” or “reducing the severity of”. The term “prevention of a certain condition” also includes “treatment of a person at (increased) risk of said condition”.

An infant is a child under the age of 12 months.

“Infant formula” or “follow-on formula” or “young child formula” means that it concerns a composition that is artificially made or in other words that it is a synthetic composition (i.e., the synthetic composition is not breast milk). Hence the nutritional composition that is administered is an artificial infant formula or an artificial follow-on formula or an artificial young child formula or a synthetic infant formula or a synthetic follow-on formula or a synthetic young child formula.

Infant formula refers to nutritional compositions, artificially made, intended for infants of 0 months to about 4 months to 6 months of age and are intended as a substitute for human milk. Typically, infant formulas are suitable to be used as sole source of nutrition up to 6 months of age. Such infant formulas are also known as starter formula. Preferably an infant formula is a nutritional composition intended for infants of 0 months to 6 months of age.

Follow-on formulas are nutritional compositions for infants starting at about 4 months to 6 months of life up to 12 months of life and are intended to be complete or supplementary feedings for infants that start weaning on other foods. Preferably follow-on formulas are nutritional compositions intended for infants from 6 months up to 12 months of age. Infant formulas and follow-on formulas are subject to strict regulations, for example for the EU regulations no. 2016/127 and 2016/128.

A young child is a child aged between one and three years, i.e. between 12 and 47 months of age, also called a toddler.

Young child formula refers to nutritional compositions, artificially made, intended for children of 12 months up to 48 months of age, which are intended as sole or supplementary feedings for children.

“Nutritional composition” means a substance or formulation that satisfies at least a portion of a subject's nutrient requirements. The nutritional composition according to the present invention is preferably selected from an infant formula, a follow-on formula, and a young child formula. This means that the present nutritional composition is not human milk. Alternatively, the term “formula” means that it concerns a composition that is artificially made or in other words that it is synthetic. Hence in one embodiment the nutritional composition is selected from an artificial infant formula, an artificial follow-on formula and an artificial young child formula or a synthetic infant formula, a synthetic follow-on formula and a synthetic young child formula.

As used herein, “hypoallergenic” refers to a composition having little or reduced likelihood of causing an allergic response.

The term HMO or HMOs refer to human milk oligosaccharide(s). HMOs are complex carbohydrates found in human breast milk ((Urashima et al.: Milk Oligosaccharides. Nova Science Publisher (2011); Chen Adv. Carbohydr. Chem. Biochem. 72, 113 (2015)). These carbohydrates are resistant to enzymatic hydrolysis by digestive enzymes. Each oligosaccharide is based on a combination of glucose, galactose, sialic acid (N-acetylneuraminic acid), fucose and/or N-acetylglucosamine. HMOs can be divided in neutral or non-acidic HMOs which can either be fucosylated or non-fucosylated, and acidic HMOs that have at least one sialyl residue in their structure. In the context of the present invention lactose is not regarded as an HMO species. HMOs can be manufactured by means known in the art.

As used herein, the term “degree of polymerization” (DP) means the number of monomer units joined together in a poly- or oligomer.

As used herein, the term “soluble” in the case of a polysaccharide, fiber, or oligosaccharide means that the substance is at least 50% soluble according to the method described by L. Prosky et al., J Assoc. Off. Anal. Chem. 71, 1017-1023 (1988).

As used herein, the term “fermentable” refers to the ability to undergo (anaerobically) decomposition by microorganisms in the colon into smaller molecules, in particular short-chain fatty acids and lactate. Fermentability can be determined, for example, by the method described in Am. J. Clin. Nutr. 53, 1418-1424 (1991).

Immune maturation as used herein refers to development or skewing of the immune system characterized by an increase in Th1 over Th2 balance that is associated with a healthy adult phenotype and important in early life immune maturation. A healthy adult phenotype is contrasted with a default Th2 phenotype that is characteristic of atopy including food allergy, allergic asthma, or certain types of autoimmune disease. Immune maturation can be assessed by measuring relative contributions of at least one Th1 marker and at least one Th2 marker, in particular the ratio of IFN-gamma (IFN-γ) to IL-13 is used herein as a marker of the Th1 to Th2 balance.

A T helper 1 response (Th1) as used herein refers to an objectively measurable manifestation of a Th1 immune phenotype. Th1 markers include, without limitation, certain cytokines including interferon gamma (IFN-γ) when secreted by CD4+ T helper cells. Methods for measuring Th1 cytokines are well known in the art and can include, without limitation, appropriate cytokine-specific or isotype-specific enzyme-linked immunosorbent assay (ELISA), bioassay, quantitative reverse transcriptase-polymerase chain reaction, and the like.

A T helper 2 response (Th2) as used herein refers to an objectively measurable manifestation of a Th2 immune phenotype. Th2 markers include, without limitation, certain cytokines including interleukin 13 (IL-13) when secreted by CD4+ T helper cells. Methods for measuring Th2 cytokines are well known in the art and include methods mentioned for measuring Th1 cytokines.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

The present invention concerns a nutritional composition comprising 3-FL and a source of butyrate.

### 3-Fucosyllactose

The nutritional composition of the present invention comprises 3-fucosyllactose (3-FL). 3-FL was found to improve the immune function. 3-FL as used herein has an immunomodulatory role. In the context of the invention 3-FL is not considered the source of butyrate; although 3-FL may be fermented by the gut microbiome it gives rise to relatively low levels of butyrate. In some aspects, improving immune function involves decreasing Th2 type IL-13 secretion. Fucosyllactose (FL) is a non-digestible oligosaccharide present in human milk. It is not present in bovine milk. It consists of three monosaccharide units, fucose, galactose and glucose linked together. Lactose is a galactose unit linked to a glucose unit via a beta 1,4 linkage. A fucose unit is linked to a galactose unit of a lactose molecule via an alpha 1,2 linkage (2′-fucosyllactose, 2′-FL, Fucα1-2Galβ1-4Glc) or via an alpha-1,3 linkage to the glucose unit of a lactose (3-Fucosyllactose, 3-FL, Galβ1-4(Fucα1-3)Glc).

3-FL, preferably Galβ1-4(Fucα1-3)Glc, is commercially available, for instance from Sigma-Aldrich. Alternatively, it can be isolated from human milk, for example as described in Andersson & Donald, 1981, J Chromatogr. 211:170-1744, or produced by genetically modified micro-organisms, for example as described in Huang et al, 2017, Metabolic Eng. 41: 23-38.

Preferably, a nutritional composition according to the invention comprises 10 mg to 1 g 3-FL per 100 ml, more preferably 20 mg to 0.5 g, even more preferably 20 mg to 0.2 g 3-FL per 100 ml. Based on dry weight, the present nutritional composition preferably comprises 0.075 wt. % to 7.5 wt. % 3-FL, more preferably 0.11 wt. % to 3.75 wt. % 3-FL, even more preferably 0.15 wt. % to 1.5 wt. % 3-FL. Based on energy, the present nutritional composition preferably comprises 0.015 g to 1.5 g 3-FL per 100 kcal, more preferably 0.023 g to 0.075 g 3-FL per 100 kcal, even more preferably 0.03 g to 0.3 g 3-FL per 100 kcal. A lower amount of 3-fucosyllactose will be less effective in modulating the immune response, promoting immune maturation, or preventing and/or treating allergy, whereas a too high amount will result in unnecessary high costs of the product.

In an aspect of the invention the nutritional composition does not comprise 2′-FL.

### Butyrate

The present nutritional composition contains dietary butyrate or non-digestible saccharides selected from non-digestible polysaccharides [NDP], non-digestible oligosaccharides [NDO] or mixtures thereof capable of undergoing microbial fermentation in the gastrointestinal tract to produce butyrate and that are suitable for use in human subjects, preferably infants or young children that suffer from or are at risk of allergy, in particular food allergy. Said non-digestible saccharides selected from NDP, NDO or mixtures thereof are fermented by the microbiota and as a result provide butyrate in the gastrointestinal tract. It was found that 3-FL together with a separate source of butyrate improves the Th1/Th2 balance. 3-FL alone does not result in a high level of butyrate in the intestine. In the context of the invention 3-FL is not a source of butyrate, the beneficial effect of 3-FL is obtained when providing 3-FL together with a separate source of butyrate. In the context of the invention 3-FL is also not the source of butyrate as fermentation would result in diminished availability of the amount of 3-FL. In the context of the invention the source of butyrate may thus be a direct source of butyrate containing butyrate or an indirect source that promotes the production of butyrate in the gut through the fermentation by the gut microbiome.

### Dietary Butyrate

The nutritional composition preferably comprises between 0.3 and 5 wt. % butyric acid based on based on weight of total fatty acyl chains, preferably between 0.6 and 5 wt. %, even more preferably between 1 and 5 wt. %. The present nutritional composition in an embodiment comprises tributyrin (i.e., triglyceride with 3 butyric acid chains attached to the glycerol backbone via ester bonds). In a further preferred embodiment, the nutritional composition may comprise synthetic triglycerides such as those described in EP3897608 B1 wherein single butyrate moiety containing triglycerides are described.

Preferably the nutritional composition contains 0.075 to 1.3 wt. % butyrate based on dry weight of the composition, preferably between 0.15 and 1.3 wt. % and more preferably between 0.25 and 1.3 wt. %. Alternatively, the nutritional composition comprises 0.015 to 0.25 g butyrate per 100 kcal, preferably 0.03 to 0.25 g butyrate per 100 kcal, and more preferably 0.05 to 0.25 g butyrate per 100 kcal. When the nutritional composition is a liquid, the composition preferably contains 0.01 to 0.175 g butyrate per 100 ml, more preferably 0.02 to 0.175 g butyrate per 100 ml, and more preferably 0.035 to 0.175 g butyrate per 100 ml.

The dietary butyrate can be supplied by any suitable source known in the art. Non-limiting sources of dietary butyrate includes animal source fats and derived products, such as but not limited to anhydrous milk fat, butter oil, and microbial fermentation derived products, such as but not limited to yogurt and fermented buttermilk. In the context of the invention well known other sources of butyrate such as milk, butter fat, butter, butter milk, butter serum and cream are less preferred because of the presence of milk proteins in said products. In some embodiments, the dietary butyrate is synthetically produced.

In embodiments where the dietary butyrate is synthetically produced, the chemical structure of the dietary butyrate may be modified as necessary. Further, the dietary butyrate produced synthetically can be purified by any means known in the art to produce a purified dietary butyrate additive that can be incorporated into the nutritional compositions disclosed herein. The dietary butyrate may be provided by dairy lipids and/or triglyceride bound forms of butyrate.

In some embodiments, the dietary butyrate may comprise butyrate salts, for example, sodium butyrate, potassium butyrate, calcium butyrate, magnesium butyrate, and combinations thereof. In certain embodiments, dietary butyrate comprises a suitable butyrate salt that has been coated with one or more fats or lipids. In certain embodiments wherein the dietary butyrate comprises a fat-coated butyrate salt, the nutritional composition may be a dry-powdered composition into which the dietary butyrate is incorporated. Preferably the dietary butyrate is supplied as part of a triglyceride. This is advantageous because butyrate is volatile (and malodorous) when provided in free or salt form. In triglyceride form the butyrate will be released in and after the stomach due to the action of lipases.

In a preferred embodiment, the weight ratio of 3-FL to dietary butyrate is in the range of 10:1 to 1:10, preferably 5:1 to 1:5, more preferably 3:1 to 1:3.

### Non-Digestible Saccharides

In some embodiments the nutritional composition comprises non-digestible saccharides selected from non-digestible polysaccharides [NDP], non-digestible oligosaccharides [NDO] or mixtures thereof as source of butyrate. The non-digestible saccharides selected from non-digestible polysaccharides [NDP], non-digestible oligosaccharides [NDO] or mixtures thereof as source of butyrate may optionally further comprise HMOs. The non-digestible polysaccharides [NDP], non-digestible oligosaccharides [NDO] or mixtures thereof are not digested in the intestine by the action of digestive enzymes present in the human upper digestive tract, but capable of undergoing microbial fermentation in the gastrointestinal tract to produce butyrate. Together with 3FL the non-digestible saccharides provide for the beneficial effects according to the invention.

A preferred source of butyrate are non-digestible saccharides [NDS] capable of undergoing microbial fermentation in the gastrointestinal tract to produce butyrate. In the context of the invention, NDS for use in the composition are defined as NDS or dietary NDS blends that are capable of undergoing microbial fermentation in the gastrointestinal tract to produce butyrate. In a preferred aspect the NDS are non-digestible oligosaccharides.

The dietary NDS that may serve as a substrate to provide butyrate, preferably produce acetate, butyrate, propionate, and hexanoate, more preferably said fibers allow to increase the butyrate production in the gastrointestinal tract.

In a preferred embodiment the fermentation of dietary NDS beneficially results in a fatty acid production wherein butyrate is produced. Fermentation profiles of dietary NDS from either a single source of fibers or blend of fibers, can be assessed using fermentation assays known in the art. Exemplary ways to assess the fermentation of NDS include using in NDS in fermentation models with fecal sample pools of healthy adults or infants or young children with exposure to NDS for at least 24 hours at 37° C. under anaerobic conditions.

NDP as used herein refers to dietary non-digestible polysaccharides having a degree of polymerization of more than 10.

In a preferred embodiment the NDS are plant-derived, crustacean-derived and/or microorganism derived, preferably plant-derived. As used herein “derived from” refers to the natural source of the NDS. Micro-organism-derived NDS refers to NDS naturally produced by microorganism, such as for example xanthan gum. As used herein “derived from” does thus not refer to the production source when NDS are for example derived from or produced in genetically modified expression vectors.

In a preferred embodiment the NDS that may serve as a substrate to provide butyrate do not contain lactose in their molecular structure and/or backbone.

Exemplary dietary NDS for use according to the invention include non-digestible polysaccharides [NDP] such as hydrolyzed or low viscosity pectin, fructo-polysaccharides, oat beta-glucan, soy fiber, resistant starch, acacia gum, cellulose, arabinoxylan, (partially hydrolysed) guar gum, xanthan gum, locust bean gum and/or resistant starch. The NDP are fermentable and provide fatty acids, in particular beneficial amounts of butyrate.

In particular XG and LBG were NDP found capable of undergoing microbial fermentation in a fermentation model to produce beneficial amounts of butyrate. The additional advantage of XG and LBG is that in particular food allergic infants and young children often suffer from reflux and/or regurgitation and therefore benefit from thickened nutritional compositions, the latter NDP also serve as thickeners in nutritional compositions.

A suitable type of NDP is long-chain fructo-polysaccharides (IcFOS) which has an average degree of polymerization above 10, typically in the range of 10-100, preferably 15-50, most preferably above 20. A preferred type of long-chain fructo-polysaccharides is Raftilin HP having an average DP above 10.

In case the nutritional composition is based on free amino acids, the preferred NDO are not milk derived. Preferably the non-digestible oligosaccharide in such a formula is IcFOS. An exemplary NDS capable of being fermented is inulin or IcFOS.

In a further preferred embodiment, the composition according to the invention comprises a mixture of dietary NDS capable of undergoing microbial fermentation in the gastrointestinal tract to produce butyrate and dietary butyrate. Preferably the mixture of dietary NDS and dietary butyrate contains at least 50 wt % of dietary NDS, more preferably the weight ratio NDS to dietary butyrate in the mixture is in the range from 80:20 to 50:50, more preferably 75:25 to 60:40.

### Non-Digestible Oligosaccharide

Preferably nutritional compositions of the invention comprise NDS including one or more non-digestible oligosaccharides (NDOs). In a preferred aspect of the invention the NDS capable of undergoing microbial fermentation in the gastrointestinal tract to produce butyrate are NDO. These NDO may help in improving gastrointestinal disorders, often observed in allergic infants, and have an anti-allergic effect by improving the microbiota and gut barrier.

Advantageously and most preferred, the non-digestible oligosaccharide is water-soluble (according to the method disclosed in L. Prosky et al, J. Assoc. Anal. Chem 71: 1017-1023, 1988) and is preferably oligosaccharide with a degree of polymerization (DP) of 2 to 10. In a preferred embodiment the NDOs according to the invention having a DP of 2 to 10 further comprise at least NDOs having a DP of 7 or higher.

The dietary NDS selected from NDPs, NDOs and optionally additionally comprising HMOs are not digested in the intestine by the action of digestive enzymes present in the human upper digestive tract (small intestine and stomach). For example, glucose, fructose, galactose, sucrose, lactose, maltose, and the maltodextrins are considered digestible. The oligosaccharide raw materials may comprise monosaccharides such as glucose, fructose, fucose, galactose, rhamnose, xylose, glucuronic acid, GalNac etc., but these are not part of the oligosaccharide.

The NDOs according to the invention are fermented by the human intestinal microbiota. The non-digestible oligosaccharide is preferably prebiotic. The NDOs according to the invention are preferably plant-derived, crustacean-derived and/or microorganism derived, preferably plant-derived. The NDO is preferably selected from the group consisting of short chain fructo-oligosaccharides, non-digestible dextrin, polydextrose, xylooligosaccharides, arabino-oligosaccharides, arabinogalacto-oligosaccharides, arabinoxylan-oligosaccharides, gluco-oligosaccharides, glucomanno-oligosaccharides, galactomanno-oligosaccharides, mannan-oligosaccharides, chito-oligosaccharides, uronic acid oligosaccharides, and mixtures thereof.

In a preferred embodiment the non-digestible oligosaccharide is not a galacto-oligosaccharide. Generally, galacto-oligosaccharides are derived from lactose, which is not a preferred source for the use according to the invention in the treatment and/or prevention of food allergy.

In one embodiment the nutritional composition comprises short-chain fructo-oligosaccharides. The short-chain fructo-oligosaccharides preferably have an average DP in the range of 2-8, preferably 3-7, i.e., are short-chain short-chain fructo-oligosaccharides in the context of the invention. scFOS may be inulin hydrolysate products having an average degree of polymerization within the aforementioned (sub-) ranges; such scFOS products are for instance commercially available as Raftilose P95 (Orafti) or with Cosucra. scFOS alternatively can be enzymatically synthesized from sucrose by a fructosyltransferase.

The nutritional composition may in an embodiment contain a mixture of two or more types of NDSs, preferably a mixture of two NDOs or a mixture of an NDO and an NDP. In case the NDS comprises or consists of a mixture of two distinct saccharides, one non-digestible saccharide may be short-chain NDO as defined above, and one non-digestible saccharide may be long-chain NDP as defined above. Most preferably, short-chain oligosaccharides and long-chain polysaccharides are present in a weight ratio short-chain to long-chain in the range of 1:99-99:1, more preferably 1:1-99:1, more preferably 4:1-97:3, even more preferably 5:1-95:5, even more preferably 7:1-95:5, even more preferably 8:1-10:1, most preferably about 9:1. Suitable mixtures include mixtures of long-chain fructo-oligosaccharides with short-chain fructo-oligosaccharides. Hence, in one embodiment, short-chain fructo-oligosaccharides and long-chain fructo-oligosaccharides are present in a weight ratio short-chain to long-chain in the range of 1:99-99:1, more preferably 1:1-99:1, more preferably 4:1-97:3, even more preferably 5:1-95:5, even more preferably 7:1-95:5, even more preferably 8:1-10:1, most preferably about 9:1.

In case the formula is based on free amino acids, the preferred NDS are not milk derived. Preferably the NDS in such a formula is a mixture of short-chain and long-chain fructo-oligosaccharides.

In particular it was found that a mixture of long-chain fructo-oligosaccharides and short-chain fructo-oligosaccharides, showed an advantageous butyrate production in a fecal fermentation assay. Hence, in combination with IcFOS, another suitable type of oligosaccharide is short-chain fructo-oligosaccharide which has an average degree of polymerization (DP) of less than 10, preferably at most 8, preferably in the range of 2-7.

The nutritional composition preferably comprises 0.05 to 20 wt % of said NDS, more preferably 0.5 to 15 wt %, even more preferably 1 to 10 wt %, most preferably 2 to 10 wt %, based on dry weight of the nutritional composition. When in liquid form, the nutritional composition preferably comprises 0.01 to 1.0 g non-digestible saccharide, more preferably 0.25 to 0.8 g, even more preferably comprises at least 0.4 g/100 ml ready to drink nutritional composition, preferably from 0.4 to 0.8 g, more preferably from 0.6 to 0.8 g/100 ml ready to drink nutritional composition.

In a particular embodiment the use of NDSs or NDS blends is preferred. Exemplary non-digestible saccharide blends in the context of the present invention are blends that are fermentable and beneficially provide butyrate in the gut. An exemplary NDS blend is a mixture that comprises scFOS and IcFOS in a 9:1 ratio.

In a further embodiment an exemplary NDS blend providing butyrate comprises at least a) inulin, b) resistant starch and/or c) soluble soy polysaccharides. The blend is a mixture of NDO and dietary fibers that is fermentable and provides fatty acids, in particular beneficially provides butyrate. A further exemplary non-digestible saccharide blend comprises a) FOS, b) inulin, c) soy fiber, d) resistant starch, e) acacia gum and/or f) cellulose. The blend is fermentable and provides fatty acids, in particular at beneficial amounts of butyrate. A further exemplary non-digestible saccharide blend comprises a) arabinoxylan, b) oat beta-glucan, c) pectin, and/or d) resistant starch.

### HMOs

The non-digestible saccharides providing for a source of butyrate may in some embodiments in addition to NDPs and/or NDOs optionally further comprise human milk oligosaccharides [HMOs]. Said HMOs are NDOs that are not digested in the intestine by the action of digestive enzymes present in the human upper digestive tract yet capable of undergoing microbial fermentation in the gastrointestinal tract. HMO refers to those oligosaccharides that are present in human milk, and falls into the conventional definition adopted by any person in the art. The HMOs comprise, but are not limited to, fucosylated oligosaccharides (for example diFL, 2FL), N-acetylated oligosaccharides (for example lacto-N-tetraose (LNT), lacto-N-neotetraose (LNnT)) or sialylated oligosaccharides.

In some embodiments, HMOs such as 2′-FL may be provided, 2′-FL is fermentable and capable of providing or enhance SCFA production including butyrate.

In the context of the invention the source of butyrate is a separate source of butyrate that is not 3FL. 3FL is an HMO that upon microbial fermentation does not provide significant amounts of butyrate. To obtain the beneficial immunological effects according to the invention 3FL has to be combined with a separate source of butyrate; said source of butyrate may be dietary butyrate and/or NDS selected from NDP and/or NDO and optionally further comprising one or more HMOs.

### Protein Components

The protein source in the nutritional composition of the present invention is suitable for use in human subjects, preferably infants or young children that are at risk of and/or suffer from food allergy, more in particular an allergy for dietary protein, even more in particular cow's milk protein or hen's egg allergy. The nutritional composition according to the invention is a hypoallergenic nutritional composition. Therefore, the protein, or protein source, is hydrolysed protein, preferably extensively hydrolyzed protein, free amino acids, or a combination thereof.

The present invention advantageously concerns a composition, and use thereof, wherein the protein source preferably provides 7 to 20% of the total calories of the composition, more preferably the protein source provides 8 to 17% of the total calories, even more preferably the protein source provides 9 to 15% of the total calories of the composition. The present invention advantageously concerns a composition, and use thereof, wherein the protein source preferably provides 1.0 g to 3.5 g protein per 100 ml, more preferably the protein source provides 1.2 to 3.0 g per 100 ml, even more preferably the protein source provides 1.4 to 2.5 g/100 ml ready to drink nutritional composition. The present invention advantageously concerns a composition, and use thereof, wherein the protein source preferably provides 1.5 g to 5.2 g protein per 100 kcal, more preferably the protein source provides 1.8 to 4.5 g per 100 kcal, even more preferably the protein source provides 2.1 to 3.7 g/100 kcal. Alternatively, in the composition, and use thereof, according to the present invention, the amount of the protein source is preferably between 10 and 20 wt % based on dry weight of the total composition, preferably between 11 and 18 wt %, and even more preferably between 12 and 16 wt % based on dry weight of the total composition. Preferably extensively hydrolysed protein, free amino acids or a combination thereof is the sole protein source.

Hydrolysed protein can be derived from cow's milk protein such as whey protein or casein. An alternative source of suitable hydrolysed protein includes rice protein hydrolysates. Hydrolysed protein is typically obtained by hydrolysis with proteolytic enzymes. Extensively hydrolysed protein typically has been processed using an ultrafiltration step after the hydrolysis with proteolytic enzymes in order to remove the potentially allergenic intact protein and large peptides. Preferably the hydrolysed protein is hydrolysed whey protein. In a further preferred embodiment, the protein is extensively hydrolysed protein, preferably extensively hydrolysed whey protein.

The hydrolysed protein, preferably extensively hydrolysed whey protein, has a degree of hydrolysis (of the protein) from 16 to 50%, more preferably from 20 to 30%, even more preferably from 20 to 25%. The degree of hydrolysis is defined as the percentage of peptide bonds which have been broken down by enzymatic hydrolysis, with 100% being the total potential peptide bonds present. A too low degree of hydrolysis will deliver an undesirable high level of intact allergenic protein or allergenic peptides.

In contrast, partially hydrolysed protein typically has a degree of hydrolysis (of the protein) from 5 to 15% which makes it suitable to induce tolerance as to some extent allergenic parts of the protein remain intact. Partially hydrolysed milk protein, such as partially hydrolysed whey protein is suitable for infants at risk of cow's milk protein allergy yet is unsuitable for an infant or young child already suffering from cow's milk protein allergy. In some embodiments partially hydrolysed plant protein such as partially hydrolysed rice protein is used. Partially hydrolysed rice protein is suitable for cow's milk protein allergic human subjects. Rice protein hydrolysates suitable for infant formulas and infant formulas with rice protein hydrolysates are commercially available.

The peptide size and molecular weight distribution can be determined by routine methods known to the skilled person such as HPLC or size exclusion chromatography (SEC), in particular high performance size exclusion chromatography. Saint-Sauveur et al. “Immunomodulating properties of a whey protein isolate, its enzymatic digest and peptide fractions” Int. Dairy Journal (2008) vol. 18(3) pages 260-270 describes an example thereof. In short, the total surface area of the chromatograms is integrated and separated into mass ranges expressed as percentage of the total surface area. The mass ranges are calibrated using peptides/proteins with a known molecular mass.

In the context of the present invention, the extensively hydrolysed protein preferably comprises from 55 to 85% peptides with a molecular weight below 1000 Da, from 15 to 45% peptides with a molecular weight between 1000 and below 5000 Da and from 0 to 1% peptides or proteins with a molecular weight of 5000 Da and above, all based on total protein of the hydrolysate. In a preferred embodiment the extensively hydrolysed protein, preferably the extensively hydrolysed whey protein, comprises peptides with the following size distribution: 60-90% with a size <1 kDa, 10-40% peptides with a size of 1 to <5 kDa, 0-0.5% peptides with a size of 5 to <10 kDa, and 0-0.2% peptides with a size >10 kDa, based on the total protein of the hydrolysate.

In a preferred embodiment of the present invention the hydrolysed protein, preferably the extensively hydrolysed whey protein, comprises less than 100 mg peptides with a size above 5 kDa per gram protein hydrolysate, preferably less than 50 mg per gram protein hydrolysate, more preferably less than 10 mg per gram protein hydrolysate. Preferably the extensively hydrolysed protein, preferably extensively hydrolysed whey protein, comprises less than 30 mg peptides with a size above 10 kDa per gram protein hydrolysate, preferably less than 10 mg per gram protein hydrolysate, most preferably less than 5 mg per gram protein hydrolysate.

Preferably the hydrolysed protein, preferably extensively hydrolysed whey protein, comprises less than 0.8 microgram, more preferably less than 0.2 microgram allergenic beta-lactoglobulin per g protein. In the context of the invention, the expression ‘allergenic beta-lactoglobulin’ refers to intact or immunogenic beta-lactoglobulin and does not account for the extensively hydrolysed beta-lactoglobulin. Allergenic beta-lactoglobulin can be determined by methods as known in the art, such as ELISA.

For those infants who have such a severe allergy that extensively hydrolysed protein, such as extensively hydrolysed whey protein, as protein source is still an issue, or who suffer from multiple food allergies, advantageously the protein source consists essentially of free amino acids. Preferably, the protein source comprises all essential amino acids. The optimal amino acid profile for infant formulas is known in the art and is present in amino acid based infant formulas such as Neocate. A preferred embodiment of an amino acid composition is given in example 4.

### Probiotics

Preferably the nutritional composition comprises lactic acid producing bacteria selected from the group consisting of the genera Bifidobacterium and/or Lactobacillus, in particular Bifidobacterium. The intestinal microbiota of breastfed infants is high in bifidobacteria. Addition to the nutritional composition of one or more strains belonging to the Bifidobacterium genus will further improve the intestinal microbiota and its activity. More preferably the nutritional composition comprises Bifidobacterium breve. Such strains of the species of Bifidobacterium breve are commercially available or can be isolated from the microbiota of infants. An example of a commercially available strain is Bifidobacterium breve M-16V from Morinaga. The amount of Bifidobacterium and/or Lactobacillus is preferably 10⁴ to 10¹¹ cfu per gram dry weigh of the nutritional composition.

Butyrate is formed in the intestine by butyrate producing bacteria that can use lactate as a carbon source, for example Anaerostipes and Roseburia species. With the infant increasing in age the microbiota develops and lactate utilizing bacteria develop which metabolize the lactate formed by lactate producing bacteria such as bifidobacterial to propionate and in particular butyrate. Such a development of the metabolic profile is associated with beneficial effects. Infants with eczema were characterized by decreased levels of both isomers of lactate and increased levels of propionate and butyrate at the age of 12 weeks. However, this pattern was reversed at 26 weeks of age, with infants having eczema showing increased levels of lactate and decreased levels of propionate and butyrate. Wopereis et al Journal of Allergy and Clinical Immunology, Volume 141, Issue 4, April 2018, Pages 1334-1342.e5, Especially the presence of butyrate is thought to have a beneficial effect. Hence addition of lactate utilizing bacteria, such as Bifidobacteria, in the presence of fibers is beneficial as the lactate formed by the bifidobacteria is used by lactate utilizing bacteria to convert to butyrate in older infants and young children.

### Nutritional Composition

The nutritional composition according to the invention can be used as an infant. follow-on formula, young child formula, as a nutritional therapy, as a food for special medical purposes or as a nutritional supplement. The nutritional composition is preferably an oral composition. The nutritional composition is administered orally to, or intended to be administered orally to, a subject in need thereof, in particular to children and infants, including toddlers, preferably children up to 6 years of age, preferably infants or young children typically with an age of 0-36 months, more preferably infants 0-12 months of age.

Thus, in some embodiments, the nutritional composition is an infant formula, follow-on formula, or young child formula (also referred to as growing-up milk or toddler milk), preferably it is an infant formula or follow-on formula, most preferably an infant formula. The terms ‘infant formula’ and ‘follow-on formula’ are well-defined and controlled internationally and consistently by regulatory bodies. It recommends for nutritional value and formula composition, which require the prepared milk to contain per 100 ml not less than 60 kcal (250 kJ) and no more than 70 kcal (295 kJ) of energy. The EU, FDA and other regulatory bodies have set nutrient requirements in accordance therewith. This caloric density ensures an optimal ratio between water and calorie consumption. In the context of the application the energy provided by nutrients Is calculated using the Atwater calculation factors of 9 kcal per g lipid, 4 kcal per gram protein or gram digestible carbohydrate, 2 kcal per gram dietary fiber and zero kcal for the other components in the product.

Suitably, the nutritional composition is in a powdered form, which preferably can be reconstituted with water to form a liquid. When the nutritional composition is in a liquid form, the preferred volume administered on a daily basis is in the range of about 80 to 2500 ml, more preferably about 450 to 1000 ml per day.

The nutritional composition according to the invention comprises lipids, preferably lipids suitable for infant nutrition as known in the art. The lipids of the nutritional composition preferably provide 2.8 to 7.0 g, more preferably 4.0 to 6.0 g per 100 kcal of the nutritional composition. When in liquid form, the nutritional composition preferably comprises 1.9 to 4.7 g lipids per 100 ml, more preferably 2.7 to 4.0 g per 100 ml. Based on dry weight the present nutritional composition preferably comprises 12.5 to 40 wt % lipids, more preferably 19 to 30 wt %.

The nutritional composition according to the invention may further comprise long-chain polyunsaturated fatty acids (LC-PUFA). LC-PUFA are fatty acids wherein the acyl chain has a length of 20 to 24 carbon atoms and wherein the acyl chain comprises at least two unsaturated bonds between carbon atoms in the acyl chain. More preferably the nutritional composition comprises at least one LC-PUFA selected from the group consisting of eicosapentaenoic acid (EPA, 20:5 n3), docosahexaenoic acid (DHA, 22:6 n3), arachidonic acid (ARA, 20:4 n6) and docosapentaenoic acid (DPA, 22:5 n3), preferably the nutritional composition comprises at least DHA and EPA, more preferably DHA, EPA and ARA. Such LC-PUFA have a further beneficial effect on reducing the risk for allergy. The preferred content of LC-PUFA in the nutritional composition does not exceed 15 wt % based on total fatty acids, preferably does not exceed 10 wt %, even more preferably does not exceed 5 wt %. Preferably the nutritional composition comprises at least 0.2 wt %, preferably at least 0.25 wt %, more preferably at least 0.35 wt %, even more preferably at least 0.5 wt % LC-PUFA based on total fatty acids. The amount of DHA is preferably at least 0.2 wt %, more preferably at least 0.3 wt %, more preferably at least 0.35 wt %, even more preferably 0.35-0.6 wt % based on total fatty acids.

The nutritional composition comprises digestible carbohydrates. Typically, digestible carbohydrates that are known in the art to be suitable for use in infant nutritional compositions, for example selected from digestible polysaccharides (e.g., starch, maltodextrin), digestible monosaccharides (e.g. glucose, fructose), and digestible disaccharides (e.g. lactose, sucrose). Particularly suitable is lactose and/or maltodextrin. Preferably the nutritional composition comprises lactose. Lactose is the main digestible carbohydrate in milk and has a relatively low glycemic index. For those infants that need an amino acid-based formula, lactose is not desired as it is derived from milk. In that case preferably the nutritional composition comprises maltodextrin. Maltodextrin consists of D-glucose units connected in chains of variable length. The glucose units are primarily linked with α(1→4) glycosidic bonds and is typically composed of a mixture of chains that vary from three to 17 glucose units long. Maltodextrins are classified by DE (dextrose equivalent) and in case maltodextrin is included in the present nutritional composition as digestible carbohydrate, the maltodextrin preferably has a DE from 3 to 47, more preferably 18 to 23.

Minerals, vitamins, trace elements and other micronutrients are present as known in the art to comply with the directives on infant and follow-on formulas and food for medical purposes intended for infants.

### Application

The composition according to the present use is preferably enterally administered, more preferably orally. The present nutritional composition is preferably an infant formula, a follow-on formula, or a young child formula. Examples of a young child formula are toddler milk, toddler formula and growing up milk. More preferably the nutritional composition is an infant formula or a follow-on formula. The present composition can advantageously be applied as a complete nutrition for infants.

The nutritional composition of the present invention is preferably suitable for use in providing nutrition to food allergic human subjects and those at risk thereof. The present nutritional composition is specifically intended for use in food allergic infants and/or food allergic toddlers and/or those at risk thereof, more preferably infants. In a preferred embodiment the present nutritional compositions are specifically intended for use in food allergic infants and/or food allergic toddlers, more preferably food allergic infants.

The infant formula, follow-on formula or young child formula may be in the form of a liquid, preferably a ready-to-drink liquid, or in the form of a powder. In one embodiment the infant formula, follow-on formula or young child formula is in the form of a powder, suitable to reconstitute with water to provide a ready-to-drink infant formula, follow-on formula, or young child formula. It is to be understood that when the infant formula, follow-on formula or young child formula according to the invention is in the form of a powder, the amounts of all ingredients including 3-FL and dietary fibers and/or dietary butyrate in said formula are defined as the amounts that would be present after reconstitution of the powder with water, i.e. the amounts are defined in mg per 100 ml ready-to-drink formula.

The nutritional composition according to the invention is for use in providing nutrition to an infant or young child, preferably an infant, preferably up to 12 months of age.

The infant formula, follow-on formula, or young child formula according to the invention is for use in providing nutrition to an infant or young child, preferably an infant, preferably up to 12 months of age.

The preferred embodiments described above for the infant formula, follow-on formula, and young child formula according to the invention also apply to the present infant formula for use, follow-on formula for use and young child formula for use.

The invention further relates to a composition comprising 3-FL, dietary NDS and/or dietary butyrate or the composition according to the invention for use as a medicament. Preferably said composition is for use in improving the immune function, for use in immune maturation, for use in prevention and/or treatment of allergy, in particular food allergy. Immune maturation comprises development of the immune response characterized by an increased Th1 over Th2 balance that is associated with a healthy adult phenotype.

Preferably said composition is for use in improving the immune function, for use in immune maturation, preferably for use in reducing and/or depressing the Th2 response and improving and/or promoting the Th1 response. Preferably said composition is for use in providing a Th1:Th2 balance wherein the balance is shifted to a Th1 response. In an embodiment the Th1 to Th2 balance comprises the IFN-γ to IL-13 cytokine release ratio.

In a particular embodiment, the composition is for administration to human subjects, in particular infants, at risk of developing allergy or suffering from allergy, especially cow's milk protein or hen's egg allergy. Infants that are known to be at risk of developing allergy include infants born from at least one parent suffers from, or has suffered from, atopic disorders (e.g. eczema) and/or allergy, most in particular from CMA.

Effects on the immune system according to the invention will have an effect on inducing oral tolerance to allergens. In context of the invention, it is understood that while preventing food allergies focuses on reducing the risk of developing allergies, inducing oral tolerance is about training the immune system to tolerate specific allergens.

Effects both on IL-13 as well as IFN-γ indicated an unexpected and improved modulation in responsiveness of the immune cells in the presence of a combination of 3-FL and butyrate. As the nutritional compositions of the invention has an improved effect on decreasing the Th2 response and increasing the Th1 response, it thereby will prevent and/or treat allergy. The effects indicate an improved effect on tolerance induction and may thus modulate and/or promote oral tolerance induction leading to a prevention from food allergy or a faster outgrowth of food allergy by regulating type 2 allergic response. 3-FL in combination with a source of butyrate is capable of suppressing food allergen-induced type 2 responses, while enhancing type 1 immunity.

The nutritional composition according to the invention is preferably for use in providing nutrition to an infant or young child, preferably an infant, that has an increased risk of developing food allergy or suffers from food allergy. The food allergic subject or subject at risk thereof is preferably a food allergic infant and/or toddler. In an embodiment the subjects have a food allergy or are at risk thereof, in particular an allergy for a dietary protein, more preferably suffer from cow's milk protein or hen's egg allergy. The nutritional composition of the present invention is preferably used to prevent and/or treat food allergy, more preferably CMA or hen's egg. The nutritional composition of the present invention is preferably used in the dietary management of food allergy and the risk of food allergy, more preferably the induction of tolerance to cow's milk protein, the prevention of CMA.

### EXAMPLES

The invention is further illustrated by the following examples.

### Example 1: In Vitro Assessment of the Effect of 3-FL and Butyrate on Immune Function

The interaction between butyrate and the most common fucosylated human milk oligosaccharides, 2′-fucosyllactose (2′-FL) and 3-fucosyllactose (3-FL), on intestinal epithelial function upon ovalbumin (OVA)-induced type 2 activation of intestinal epithelial cells (IEC) and subsequent dendritic cell (DC) maturation and T-cell functioning was assessed in a sequential mucosal co-culture model.

By using intestinal epithelial cells (IEC)/dendritic cells (DC) and DC/T-cell cocultures, the effects of epithelial preincubation with 2′-FL or 3-FL and/or butyrate was assessed in the conditions where the IEC were also challenged by ovalbumin (OVA) to induce epithelial inflammation. This experimental model was recently used to demonstrate the differential immunomodulatory effects of 2′-FL and 3-FL alone [M. Zuurveld et al., J. Innate Immun., 2022.

The human colon adenocarcinoma HT-29 cell line (passages 148-156) was used to model IEC. Confluent HT29 cells in transwell plate were incubated with 2′-FL or 3-FL (0.1% w/v), and/or butyrate (0.5 mM) for 24 h. Thereafter, medium was refreshed containing new HMOs and butyrate and designated conditions were exposed to ovalbumin (100 μg/mL) for another 24 h. Basolateral medium was collected for cytokine analysis and after washing the HT29 cells with new medium, the monocyte-derived (moDCs) were added to the basolateral compartment for 48 h. moDCs were collected and subsequently cocultured with naïve T cells for 5 days. Supernatants were collected from the different cell culture steps and were analyzed for chemokine and cytokine secretion with Enzyme-Linked Immunosorbent Assays (ELISA).

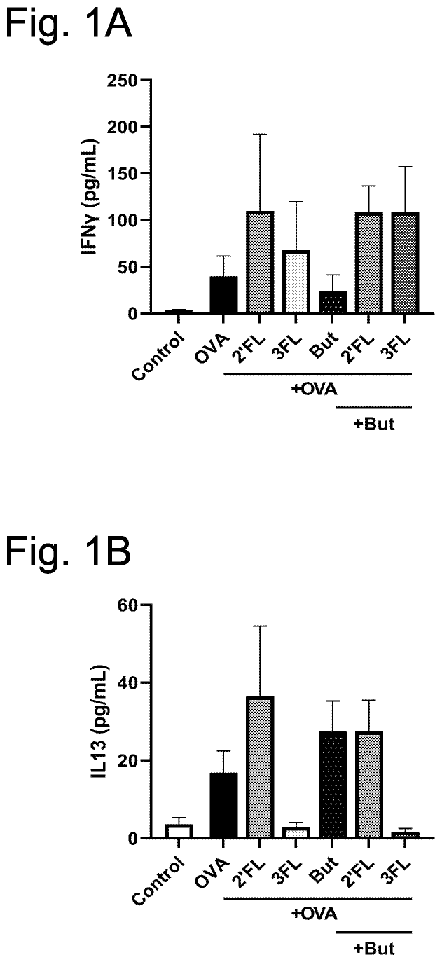

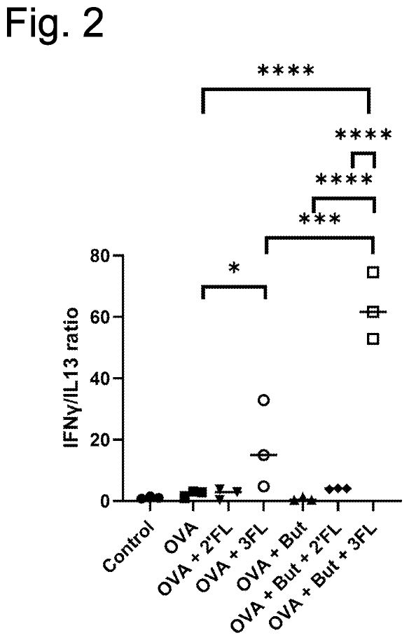

Priming DC by OVA-exposed IEC (OVA as allergic inflammatory trigger), resulted in slightly elevated IFNγ secretion, which tended to further increase if IEC were preincubated with 2′-FL or 3-FL in combination with butyrate (FIG. 1A). In addition, priming DC by OVA-exposed IEC, resulted in enhanced IL-13 secretion during T cell coculture (FIG. 1B), and here preincubation of OVA-IEC with butyrate alone had no significant effects on downstream OVA induced T helper cell cytokine secretion (FIG. 1B). However, 3-FL preincubation prevented OVA-IEC/DC mediated IL-13 secretion by T cells, and this effect was still observed when 3-FL was combined with butyrate (FIG. 1B). This was not the case for 2′-FL, where the enhanced IL-13 secretion was not affected by the combination with butyrate. Therefore, under these inflammatory conditions butyrate mainly affected the immunomodulatory characteristics of 3-FL, while the effects of 2′-FL remained present. However, in both cases combined incubation of either HMOS with butyrate during IEC/DC culture, downstream tended to enhance IFN-γ secretion by the T cells, but only the 3-FL combination with butyrate maintained low levels of IL-13 and led to shifting the Th1/Th2 response ratio in a favorable direction for allergy prevention (FIG. 2).

Our results reveal distinct immunomodulatory effects in the OVA-exposed in vitro model when butyrate preincubation of IEC/DC was combined with either 2′-FL or 3-FL. A general boost in both inflammatory and regulatory cytokine secretion was observed in the presence of 2′-FL, which remained present when combination with butyrate was tested, but here type 1 immunity was further promoted. So, although an interaction between butyrate and 2′-FL was hypothesized and observed at the level of IEC/DC interaction in this OVA induced mucosal immune activation model, the effects observed after combined 2′-FL+butyrate preincubation corresponded to a great extent with the effects observed after 2′-FL preincubation alone.

Nonetheless, combined preincubation with butyrate and 3-FL enhanced secretion of IL6 and TGFβ significantly by the primed DC, which was not observed for the separate butyrate or 3-FL preincubations in the presence of OVA (data not shown). Also, the secretion of IFN-γ tended to increase in the combination 3-FL+butyrate which was not observed for the separate butyrate or 3-FL preincubation, while maintaining very low IL-13 levels.

Overall, these in vitro studies indicated that 3-FL in combination with butyrate may be capable of suppressing OVA induced type 2 responses, by skewing the immune balance in favor of the type 1 over type 2 response (ratio IFN-γ/IL-13). Overall, it was thus found that 3-FL in combination with butyrate is capable of suppressing OVA-induced type 2 responses, while enhancing type 1 immunity.

### Example 2: Infant Formula with Extensively Hydrolysed Whey Protein, 3FL and Fibers as Source of Dietary Butyrate Suitable for Allergic Infants and Infants at Risk

A packed, powdered infant formula that that after reconstitution with water according to instructions on the pack contains per 100 ml (13.46 g powder in end volume of 100 ml):
66 kcal
1.6 g protein (extensively hydrolysed whey protein)
7.1 g digestible carbohydrates (mainly lactose)
3.4 g fat (vegetable oils, fish oil)
0.4 g Prebiotics: scFOS/IcFOS in a 9:1 ratio (source RaftiloseP95 and Raftiline HP)
0.1 g 3-FL (Chr. Hansen)

Minerals vitamins trace elements and other micronutrients as according to directives.

### Example 3: Follow-On Formula with Extensively Hydrolysed Whey Protein, 3′FL and Fibers as Source of Dietary Butyrate Suitable for Allergic Infants and Infants at Risk

A packed, powdered follow-on formula that that after reconstitution with water according to instructions on the pack contains per 100 ml (14.44 g powder in end volume of 100 ml):
68 kcal
1.6 g protein (extensively hydrolysed whey protein
7.8 g digestible carbohydrates (mainly lactose) and
3.2 g fat (vegetable oils, fish oil)
0.7 g Prebiotics: scFOS/IcFOS in a 9:1 ratio (source RaftiloseP95 and Raftiline HP)
20 mg 3-FL (Chr. Hansen)

Minerals vitamins trace elements and other micronutrients as according to directives.

### Example 4: Infant Formula with Free Amino Acids, 3-FL and Dietary Butyrate Suitable for Allergic Infants and Infants at Risk

A packed, infant formula that that after reconstitution with water, according to instructions on the pack contains per 100 ml (14.7 g powder in end volume of 100 ml):
67 kcal
1.8 g protein equivalent (free amino acids) L-Alanine, L-Arginine, L-Aspartic acid, L-Cystine, L-Glutamine, Glycine, L-Histidine, L-Isoleucine, L-Lysine, L-Methionine, L-Phenylalanine, L-Proline, L-Serine, L-Threonine, L-Tryptophan, L-Tyrosine, L-Valine, L-Carnithine
7.3 g digestible carbohydrates (mainly dried glucose syrop) and including 1.0 g waxy corn starch.
3.4 g fat ((of which about 50 wt. % bovine milk fat, the remainder being vegetable oils, fish oil and microbial oil). Based on total fatty acids the amount of butyric acid is 1.48 wt. %, the amount of arachidonic acid is 0.52 wt. %, the amount of eicosapentaenoic acid is 0.11 wt. %, the amount of docosahexaenoic acid is 0.52 wt. %)
0.7 g Prebiotics: scFOS/IcFOS in a 9:1 ratio (source RaftiloseP95, and Raftiline HP)
0.1 g 3-FL (Chr. Hansen)
Minerals, vitamins, trace elements and other micronutrients as according to directives for infant formula.

### Example 5: Rice Protein Hydrolysate Based Infant Formula

A rice protein hydrolysate infant formula in powder form, packed with on the package instructions for reconstitution with water and an indication that it is for the use for the dietary management of infants of 0-12 months that suffer from cow's milk allergy.

About 13.5 g of powder is reconstituted with water to 100 ml of formula.

Per 100 g powder the composition comprises:
495 kcal
12.4 g protein equivalent, rice protein hydrolysate, L-Trp, L-Tyr, L-Ile
50.6 g digestible carbohydrates (mainly maltodextrin)
26.0 g lipid (mainly vegetable lipid and containing microbial oil delivering 0.51 wt % arachidonic acid, 0.51 wt % docosahexaenoic acid based on total fatty acids
4.6 g non-digestible oligosaccharide mixture consisting of
   4.0 scFOS (source Raftiline HP)
   0.45 g IcFOS (source Raftiline HP)
   0.45 g 2′fucosyllactose (Chr Hansen)
   0.2 g 3-fucosyllactose
minerals, trace elements, vitamins, and other micronutrients according to international directives for infant formulas and FSMP regulation.

### Example 6—Fermentation Profile Xanthan Gum, Locust Bean Gum and Non-Digestible Oligosaccharides scGOS/IcFOS

Fecal samples were collected from a formula fed infant (5.5 months of age) and from a breastfed infant (3 months of age). The infants were without gastrointestinal problems and did not use antibiotics in the last month. Fecal samples were pooled, homogenized, divided in smaller aliquots, and mixed with glycerol (10%) in an anaerobic cabinet. Subsequent aliquot storage was at −80° C.

The non-digestible oligosaccharides were added at a concentration of 100 mg dietary fiber (DP≥2) per 6 ml of feces suspension, see the conditions in the table 1. Source of GOS was Vivinal GOS (Friesland Campina), source of IcFOS was Raftilin HP (Orafti). Xanthan gum (Keltrol Grindsted 808 MAS-SH clear) was obtained from CP Kelco, locust bean gum (Grindsted LBG 860) from Danisco, Valencia, Spain.

TABLE 1
Conditions in the fecal slurry fermentation

| | Condition (of dietary fibers) | Ratio (wt/wt) | Label in tables |
|---|---|---|---|
| 1 | blanc | 1 | blanc |
| 2 | glucose | 1 | glucose |
| 3 | scGOS/lcFOS | 9/1 | scG/lcF 9:1 |
| 4 | scFOS/lcFOS | 9/1 | scF/lcF 9:1 |
| 5 | Xanthan Gum | 1 | XG |
| 6 | Locus Bean Gum | 1 | LBG |

For the experiment, the fecal pool was defrosted in a water bath for 20 minutes at 37° C. The fecal pool was put thereafter in the anaerobic cabinet. Feces was mixed with the fermentation medium as 1:5 in a falcon tube. Samples of this fecal suspension were taken at t=0 and 6 ml of this suspension was added to a sterile falcon tube together with the substrate of interest and mixed thoroughly. Next, 6 ml of the feces/substrate suspension was put in a dialysis tube and air was removed in the empty space. The dialysis tube was put in a 100 ml Scott bottle filled with 100 ml dialysis medium. The Scott bottles were closed and incubated at 37° C. Samples of the dialysis medium (dialysate) and fecal suspension (lumen) were taken at t=24 and t=48 hours for determination of SCFA, D- and L-lactate, and gas volume.

Fermentation medium (Mc Bain and MacFarlane) contained buffered peptone water 3.0 g/l, Yeast Extract 2.5 g/l, Tryptone 3.0 g/l, L-Cysteine-HCl 0.4 g/l, Bile salts 0.05 g/l, K₂HPO₄·3H₂O 2.6 g/l, NaHCO₃ 0.2 g/l, NaCl 4.5 g/l, MgSO₄·7H2O 0.5 g/l, CaCl₂·2H₂O 0.3 g/l, FeSO₄·7H₂O 0.005 g/l. Ingredients were added one by one in 800 ml water, pH was adjusted to 5.5±0.1 with K₂HPO₄ or NaHCO₃ and volume was filled up to 1 liter. Medium was sterilized for 15 minutes at 121° C. and put in the anaerobic cabinet at least 16 hours before use.

Dialysis medium contained K₂HPO₄·3H₂O 2.6 g/l, NaHCO₃ 0.2 g/l, NaCl 4.5 g/l, MgSO₄·7H₂O 0.5 g/l, CaCl₂·2H₂O 0.3 g/l, FeSO₄·7H₂O 0.005 g/l. pH was adjusted to 5.5±0.1 with K₂HPO₄ or NaHCO₃. Medium was not sterilized because of forming of sediment. The medium was put in the anaerobic cabinet at least 16 hours before use.

The pH was measured by immersing a 423 pH-electrode (Mettler Toledo, Columbus, OH, USA), connected to a Handy-lab pH meter (Schott Glass, Mainz, Germany), directly in a sample.

Gas volume was determined with a unit to measure pressure and volume. The bottles were shaken thoroughly before measuring.

The SCFA acetic, propionic, n-butyric, iso-butyric, n-valeric, and isovaleric acids were quantitatively determined using a Shimadzu-GC2025 gas chromatograph with a flame ionization detector. As mobile phase hydrogen was used. The levels of SCFA were determined using 2-ethylbutyric acid as an internal standard. From the peak area a calibration curve was constructed and the concentration in the samples was calculated.

Lactate was determined enzymatically using an L-lactic acid detection kit with D- and L-lactate dehydrogenase (Boehringer Mannheim, Mannheim, Germany). First, samples were centrifuged for 10 min at 13,000 rpm at 4° C., then the supernatant was heated for 10 min at 100° C. to inactivate all enzymes and then the samples were centrifuged for 10 minutes at 13,000 rpm.

### Results

Levels of isobutyric acid, valeric acid, and isovaleric acid were below detection limit. Lactic acid, predominantly L-lactic acid, was formed at t=24 h in the mixtures comprising scG/IcF.

Butyric acid, and especially propionic acid, were high when xanthan gum or locust bean gum was the sole fiber. As a result the SCFA profile showed less % of acetic acid, and higher % of butyric acid and propionic acid.

These effects were present at t=24 and 48 h. Table 2 shows the SCFA production and gas production after 48 h, a duration representative for the colonic transit time in an infant.

TABLE 2
Formation of butyrate (in μmol/g fiber) after
48 h fermentation by infant intestinal microbiota.

| | | | scG/lcF | scF/lcF | | |
|---|---|---|---|---|---|---|
| | Blanc | glucose | 9:1 | 9:1 | XG | LBG |
| Butyrate (μmol/g) | 40 | 40 | 40 | 90 | 77 | 50 |

Overall, the fermentation profile shows that the mixture of scFOS with IcFOS, LBG and particularly XG beneficially result in butyrate formation.

### Example 7—Fermentation Profile of FOS with Bifidobacterium breve with Faecal Samples of a Breast-Fed Infant of 4 Months of Age

Faecal samples were obtained from a breast fed infant of 4 months of age.

### Probiotic Strain

Frozen pellets (counted stock) of Bifidobacterium breve M-16V (Morinaga) containing a pNZ44St plasmid conferring Streptomycin resistance. This enables to follow strain by selective plating on TOS-propionate agar with streptomycin supplement. Per feeding the final dose of B. breve was 1×10⁸ cfu.

The following experimental arms were compared (sterile stock solutions):
scFOS/IcFOS: 5 g/L (10% solution) scFOS/IcFOS in a 9:1 wt/wt ratio;
scFOS/IcFOS 5 g/L scFOS/IcFOS (10% solution)+1.10⁸ cfu/ml B. breve M-16V

Growth medium: Colonic Microbiota Medium: Yeast extract 1 g/L, Ammoniumsulphate 2 g/L, K₂HPO₄ 2 g/L, NaHCO₃ 3.2 g/L, NaCl 4.5 g/L, MgSO₄·7H₂O 0.5 g/L, CysteinHCl 0.5 g/L, CaCl₂·2H₂O 0.4 g/L, Bile Salts 25 mg/L, mendione, metal and vitamin solution, haemin (10 mg/L), Mucus 2.5 g/L.

The infant faecal samples were thawed under anaerobic conditions and approximately a 4% (w/v) suspension of the fecal samples were made in Colonic Microbiota medium containing 25 mM acetate and 12 mM lactate, 25 mg/L bile acids (Sigma), 2.5 g/L porcine stomach mucin, 15 mmol/L ammonium sulphate, 1 g/l tryptone, without carbon source, adjusted to pH 5.5 (to mimic the pH of breast fed infant faces). The diluted faecal samples were homogenized, allowed to sediment for 5 minutes, then filtered over a tea sieve to remove large particles and subsequently filtered over a Millex 100 μm vacuum filter.

A Biolector Pro plate (BOH2 round well, M2P-labs) with pH optodes was filled with fecal samples. Plates were sealed with ventilated silicone foil with slits. The plate was incubated (85% moisture, 37° C., 600 rpm, anaerobic) in BioLector Pro. At T=0, respectively, 80 microliters of the sterile experimental compositions were added as bolus feeding. If appropriate a thawed pellet of M-16V was added in a concentration of 1×10⁸ CFU. The experiment was started with pH at setpoint 5.5 for infants' samples with continuous pH control. After 4 hours the experiment was paused, the faecal slurry of each well was harvested and shortly centrifuged. All under a-septic anaerobic conditions. The supernatant was frozen for further analyses while the faecal pellet was resuspended in fresh medium with carbohydrates and pipetted back in original well of BOH2 plate. The procedure was repeated again for 3 days, with every day 2 times manual feeding every 4 hours followed by automatic continuous slow carbohydrate feeding (80 μl overnight) via the BioLector system via the microfluidic system.

Faecal supernatants of the fecal fermentations were thawed and aliquoted to quantitatively determine the SCFA acetic, propionic, n-butyric, isovaleric and n-valeric acids by gas chromatography, ammonia concentration by the rapid Ammonia kit (Megazyme) and after heat inactivation D- and L-lactate by the D-lactic acid/L-lactic acid kit (R-Biopharm AG).

### Results

Butyrate production upon exposure to a mixture of scFOS with IcFOS was beneficially further increased upon exposure to the Bifidobacterium breve.

TABLE 3
Formation of fermentation end products (in mmol/L)
by breast-fed infant intestinal microbiota.

| | SCFA |
|---|---|
| | (mmol/l) |
| | Butyrate |
| scFOS/lcFOS | 2.8 |
| scFOS/lcFOS/B. breve | 8.8 |

1-16. (canceled)

17. A nutritional composition comprising digestible carbohydrates, lipids, protein, 3-fucosyllactose (3-FL), and a source of butyrate, suitable for providing nutrition to a human subject, preferably an infant or young child suffering from or at risk of developing a food allergy, preferably cow's milk protein or hen's egg allergy, wherein the protein is or essentially consists of hydrolysed protein and/or free amino acids; wherein the source of butyrate is provided in the form of dietary butyrate and/or by non-digestible saccharides capable of undergoing microbial fermentation in the gastrointestinal tract to produce butyrate; and wherein the non-digestible saccharides are selected from non-digestible polysaccharides (NDP), non-digestible oligosaccharides (NDO) or mixtures thereof, and wherein the non-digestible saccharides are plant-derived, crustacean-derived and/or microorganism-derived, wherein a young child is between 12 and 47 months.

18. The nutritional composition according to claim 17, in the form of a ready-to-drink liquid or in the form of a powder that upon reconstitution with water is a ready-to-drink liquid nutritional composition, wherein the 3-FL is present in an amount of 10 mg to 1 g per 100 mL nutritional composition.

19. The nutritional composition according to claim 17, wherein the butyrate is provided in the form of dietary butyrate and/or by non-digestible saccharides capable of undergoing microbial fermentation in the gastrointestinal tract to produce butyrate, wherein the nutritional composition comprises 10 mg to 175 mg dietary butyrate per 100 mL nutritional composition and/or 1 mg to 1.0 g non-digestible saccharides per 100 mL nutritional composition.

20. The nutritional composition according to claim 17, wherein the source of butyrate comprises dietary butyrate, wherein the sources of dietary butyrate are selected from the group consisting of food-grade tributyrin, anhydrous milk fat, butter oil, and microbial-fermentation-derived products.

21. The nutritional composition according to claim 17, wherein the source of butyrate comprises non-digestible oligosaccharides selected from the group consisting of fructo-oligosaccharides, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides, chito-oligosaccharides, uronic-acid-oligosaccharides, and mixtures thereof.

22. The nutritional composition according to claim 17, wherein the source of butyrate comprises non-digestible polysaccharides selected from the group consisting of low-viscosity pectin, (partially hydrolysed) guar gum, fructo-polysaccharides, oat beta-glucan, soy fibre, resistant starch, acacia gum, cellulose, arabinoxylan, xanthan gum, locust bean gum and/or resistant starch.

23. The nutritional composition according to claim 17, wherein the source of butyrate comprises a mixture of non-digestible polysaccharides and non-digestible oligosaccharides, preferably a mixture of short-chain fructo-oligosaccharide (scFOS) and long-chain fructo-oligosaccharides (lcFOS), more preferably in a weight ratio of 9:1, wherein the long-chain oligosaccharides have an average degree of polymerisation greater than 10.

24. The nutritional composition according to claim 17, wherein the source of butyrate comprises milk fat and/or fructo-oligosaccharides, preferably a mixture of short-chain fructo-oligosaccharides and long-chain fructo-oligosaccharides.

25. The nutritional composition according to claim 17, which is an infant formula, a follow-on formula or young-child milk.

26. The nutritional composition according to claim 17, further comprising Bifidobacterium, preferably B. breve.

27. The nutritional composition according to claim 17, wherein the protein is or essentially consists of hydrolysed whey protein or hydrolysed rice protein.

28. A method of treating or preventing food allergy in infant or young child suffering from or at risk of food allergy, comprising administering to the infant or young child a nutritional composition comprising digestible carbohydrates, lipids, protein, 3-fucosyllactose (3-FL), and a source of butyrate, wherein the protein is or essentially consists of hydrolysed protein and/or free amino acids; wherein the source of butyrate is provided in the form of dietary butyrate and/or by non-digestible saccharides capable of undergoing microbial fermentation in the gastrointestinal tract to produce butyrate; and wherein the non-digestible saccharides are selected from non-digestible polysaccharides (NDP), non-digestible oligosaccharides (NDO) or mixtures thereof, and wherein the non-digestible saccharides are plant-derived, crustacean-derived and/or microorganism-derived, wherein a young child is between 12 and 47 months.

29. The method according to claim 28, wherein the treatment or prevention of food allergy is preferably hen's-egg protein allergy or cow's-milk protein allergy, more preferably cow's-milk protein allergy.

30. Method according to claim 28, wherein treating or preventing food allergy comprises stimulating the immune function, promoting immune maturation, reducing and/or depressing the Th2 response, improving and/or promoting the Th1 response, preferably increasing the Th1:Th2 balance.

31. Method of treating or preventing food allergy in an infant or young child by modulating immune maturation, the method comprising administering to the infant or young child a nutritional composition as defined in claim 17.
